(19) United States
(12) Patent Application Publication
(10) Pub. No.: US 2012/0322829 A1
(43) Pub. Date: Dec. 20, 2012

(54) USE OF A DIPYRIDYL COMPOUND FOR TREATING ROSACEA

(75) Inventors: Irina Safonova, Nice (FR); Jean-Dominique Pierret, Valbonne (FR)

(73) Assignee: GALDERMA RESEARCH & DEVELOPMENT, Biot (FR)

(21) Appl. No.: 13/512,832

(22) PCT Filed: Nov. 26, 2010
(86) PCT No.: PCT/FR2010/052543

§ 371 Date: Aug. 10, 2012

(30) Foreign Application Priority Data
Nov. 30, 2009 (FR) 0958478

(51) Int. Cl. A61K 31/444; A61P 17/00; A61P 27/02

(52) U.S. Cl. 514/334

(57) ABSTRACT

A compound of formula (I) below:

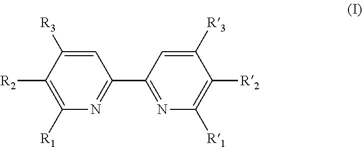

or one of the pharmaceutically acceptable salts thereof is described. Also described, are uses thereof in the treatment of rosacea.

The present invention relates to the use of a compound of formula (I) in the treatment of rosacea.

Rosacea is a chronic and progressive common inflammatory dermatosis associated with vascular relaxation. It mainly affects the central part of the face and is characterized by reddening of the face or hot flushes, facial erythema, papules, pustules, telangiectasia and occasionally ocular lesions known as ocular rosacea. In serious cases, especially in men, the soft tissue of the nose may swell and produce a bulbous swelling known as rhinophyma. Rosacea develops over several years via episodes that are worsened by various stimuli such as temperature variations, alcohol, spices, exposure to sunlight, or emotions.

Rosacea is classified into four subtypes as a function of various clinical features (Wilkin, J. et al., JAAD, 2002, 46: 584-587).

The primary features (vasomotor flushing, persistent erythema, papules and pustules, and telangiectasia) and secondary features (burning or stinging sensation, plaques, oedema, ocular manifestations, phymatous changes) of rosacea are often observed in combination. The most common modes of exteriorization or combinations of signs are temporarily regrouped into specific subtypes, which are described below. Each category comprises the minimum number of signs that are sufficient to make a diagnosis of the corresponding subtype (although the modes of exteriorization are not necessarily limited to these signs), and it is possible that patients simultaneously present features suggesting more than one subtype of rosacea.

### Subtype 1: Erythematotelangiectatic Rosacea

Erythematotelangiectatic rosacea is mainly characterized by vasomotor flushing and persistent central facial erythema. The presence of telangiectasias is common, but not essential for the diagnosis of this subtype. A central facial oedema, burning and stinging sensations, and roughness or desquamation are also occasionally observed.

A history of vasomotor flushing alone is common among patients suffering from erythematotelangiectatic rosacea.

### Subtype 2: Papulopustular Rosacea

Papulopustular rosacea is characterized by persistent central facial erythema and by transient papules and/or pustules distributed in the centre of the face. However, the papules and pustules may also affect the peri-orificial regions (i.e. the perioral, perinasal or periocular areas). The papulopustular subtype resembles acne vulgaris, except that comedones are absent. Rosacea and acne may coexist, and, besides the papules and pustules resembling rosacea, the patients concerned will also possibly have comedones. Patients suffering from papulopustular rosacea occasionally complain of burning and stinging sensations.

This subtype is often observed before or at the same time as subtype 1 (including the presence of telangiectasias). The telangiectasias risk being masked by the persistent erythema and the papules or pustules, and they have a tendency to become more visible after successful treatment of these masking components.

### Subtype 3: Phymatous Rosacea

Phymatous rosacea is manifested by thickening of the skin, irregular surface nodules and tumefaction. Rhinophyma is the most common presentation, but phymatous rosacea may affect other locations, including the chin, forehead, cheeks and ears. Among the patients suffering from this subtype, the presence of enlarged and prominent follicular openings is occasionally reported in the affected region, as are telangiectasias.

This subtype is often observed before or at the same time as subtype 1 or 2 (including the presence of persistent erythema, telangiectasias, papules and pustules). In the case of rhinophyma, these additional stigmata risk being particularly pronounced in the nasal region.

### Subtype 4: Ocular Rosacea (or Ophthalmic Rosacea)

The diagnosis of ocular rosacea must be envisaged when a patient has one or more of the following ocular signs and symptoms: teary or bloodshot appearance (interpalpebral conjunctival hyperaemia), sensation of presence of a foreign body, of burning or stinging, dryness, itching, photosensitivity, blurred vision, telangiectasias of the conjunctiva and of the eyelid margin, or erythema of the eyelid and periocular erythema. Blepharitis, conjunctivitis and irregularity of the eyelid margins are other signs that may be detected. A chalazion or a chronic staphylococcal infection manifested by a stye and whose cause is a meibomian gland dysfunction is a common sign of rosacea-related ocular disease. Some patients complain of a reduction in visual acuity, which is due to corneal complications (punctate keratitis, corneal infiltrates/corneal ulcers or marginal keratitis). By itself, the treatment of cutaneous rosacea may be without effect on the risk of lowering the visual acuity associated with ocular rosacea, and an ophthalmological approach will possibly be required.

Finally, other rarer forms of rosacea exist (variants), in particular granulomatous rosacea.

The diagnosis of ocular rosacea is most often made when cutaneous signs and symptoms are also detected. However, it is not necessary for cutaneous signs and symptoms to be present in order to make the diagnosis, and small-scale studies suggest that up to 20% of patients suffering from ocular rosacea may develop ocular signs and symptoms before cutaneous manifestations appear. Cutaneous lesions are the first to appear in about half of these patients, and manifestations of the two types occur simultaneously in a minority of them.

Rosacea generally occurs between the ages of 25 and 70, and is much more common in people with a fair complexion. It more particularly affects women, although this complaint is generally more severe in men.

Conventionally, rosacea is treated orally or topically. The three agents approved by the FDA for the topical treatment of rosacea are metronidazole, azelaic acid and sodium sulphacetamide-sulphur. There is only one oral treatment approved by the FDA for rosacea, Oracea, a controlled-release formulation based on doxycycline monohydrate (Nally, J. B. & Berson, D. S., J. Drug Dermatol., 2006, 5: 23-26; Baldwin, H. E., J. Drug Dermatol., 2006, 5: 16-21; Baldwin, H. E., Skin Therapy Letter, 2007, 12: 1-9).

However, the therapies available are merely suppressive and not curative. In addition, rosacea is sometimes resistant to treatment. It remains a chronic pathology with a typical profile of remission and exacerbation, associated with a significant psychosocial impact.

The pathogenesis of rosacea is poorly understood, and may involve several factors. These are, for example, vascular factors (abnormal vascular reactivity), immune factors, or alternatively exogenous factors such as the presence of follicular microorganisms such as bacteria and Demodex folliculorum mites (Diamantis, S. & Waldorf, H. A., J. Drug Dermatol., 2006, 5: 8-12; Wilkin, J. K., Arch. Dermatol., 1994, 130: 359-362; Buechner, S. A., Dermatology, 2005, 210: 100-108).

Furthermore, research, in particular clinical research, tends to suggest that rosacea is an inflammatory pathology.

An over-regulation of proinflammatory cytokines and matrix metalloproteinases (MMPs) contributes to a chronic vasodilatation and to a continuous degradation of the dermal matrix. Many studies describe increased expression and activity of MMPs, such as MMP-8 and MMP-9, in patients suffering from rosacea (Bonamigo et al., J. Eur. Acad. Dermatol. Venerol., 2005, 19: 646-647; Määttä, M. et al., Graefes Arch. Clin. Exp. Ophthalmol., 2006, 244: 957-962; Sobrin, L. et al., Invest. Ophthalmol. Vis. Sci., 2000, 41: 1703-1709; Afonso, A. A. et al., Invest. Ophthalmol. Vis. Sci., 1999, 40: 2506-2512).

A reduction in the expression and/or activity of certain MMPs has been associated with a therapeutic benefit in the case of rosacea.

Tetracycline derivatives, which include doxycycline, possess not only an antibiotic activity, but also anti-inflammatory properties. These anti-inflammatory effects due to doxycycline include a reduction in the production of proinflammatory cytokines, an inhibition of the expression of the NO synthase enzyme, and a reduction in the expression and/or the activity of certain MMPs, such as MMP-1, MMP-2, MMP-8, MMP-9, MMP-12 and MMP-13 (Del Rosso, J. Q. et al., JAAD, 2007, 56: 791-802; Webster, G. et al., Dermatol. Clin., 2007, 25: 133-135; Weinberg, J. M., Cutis, 2005, 75 (suppl. 4): 6-11; Golub, L. M. et al., Adv. Dent. Res., 1998; 12:12-26).

However, long-term oral treatments with tetracycline derivatives could be problematic for many reasons, in particular on account of their significant side effects. The oral administration of tetracyclines may induce disorders such as Candidal vulvovaginitis, gastrointestinal disorders, and even idiopathic intracranial hypertension (or pseudotumor cerebri) (Del Rosso, J. Q., Cutis, 2000, 66 (suppl. 4): 7-13; Bikowski, J. B., Cutis, 2000, 66 (suppl. 4): 3-6; Rebora, A., Am. J. Clin. Dermatol., 2002, 3: 489-496).

The current treatments that are available have unpleasant side effects for the patient. Furthermore, none of the existing treatments makes it possible to effectively treat and/or prevent all of the symptoms associated with rosacea. Considering the chronic nature of rosacea, an ideal treatment requires use that may be prolonged, in a safe and effective manner.

One of the advantages of topical therapy is the direct application to the skin, which avoids systemic side effects.

The objective of the present invention is, in particular, to propose an effective treatment of rosacea, which limits the effects. Preferably, this treatment is carried out topically, which avoids any systemic side effect.

One subject of the present invention is therefore compounds of formula (I) below:

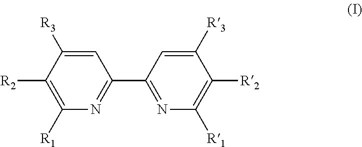

in which R1, R2, R3, R′1, R′2 and R′3 each independently represent a hydrogen atom or a linear or branched alkyl radical having from 1 to 6 carbon atoms, or the pharmaceutically acceptable salts thereof,
for the use thereof in the treatment of rosacea.

Preferably, the compound of formula (I) or salts thereof is (are) used in the treatment of at least one subtype of rosacea chosen from erythematotelangiectatic rosacea, papulopustular rosacea, phymatous rosacea and ocular rosacea.

Another subject of the present invention is the use of at least one compound of formula (I) for preparing a medicament for treating rosacea.

The compounds of formula (I) according to the invention may be used as they are, or else in the form of pharmaceutically acceptable salts.

The expression “pharmaceutically acceptable salt” is especially understood to mean a salt with a pharmaceutically acceptable inorganic acid, such as hydrochloric, sulphuric, phosphoric, nitric, carbonic, boric, sulphamic and hydrobromic acids; or else a salt with a pharmaceutically acceptable organic acid, such as acetic, propionic, butyric, tartaric, maleic, hydroxymaleic, fumaric, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, panthotenic, tannic, ascorbic and valeric acids.

The term “treatment” of or “treating” rosacea means reducing and/or inhibiting the development of rosacea and/or of the symptoms thereof.

The expression “linear or branched alkyl radical having from 1 to 6 carbon atoms” is especially understood to mean a methyl, ethyl, n-propyl, isopropyl, n-butyl, isobutyl, n-pentyl, isopentyl or n-hexyl radical.

Preferably, the radicals R1, R2, R3, R′1, R′2 and R′3 of the compounds of formula (I) each independently represent a hydrogen atom or a methyl, ethyl, propyl, isopropyl, n-propyl, butyl, isobutyl or n-butyl radical.

Preferably, at least one of the radicals R1, R2 and R3, and at least one of the radicals R′1, R′2 and R′3, each represent a methyl radical.

More preferably, the compound of formula (I) is chosen from 6,6′-dimethyl-2,2′-dipyridyl, 5,5′-dimethyl-2,2′-dipyridyl, 4,4′-dimethyl-2,2′-dipyridyl and the pharmaceutically acceptable salts thereof.

Therefore, one subject of the present invention is preferably the use of at least one compound chosen from 6,6′-dimethyl-2,2′-dipyridyl, 5,5′-dimethyl-2,2′-dipyridyl, 4,4′-dimethyl-2,2′-dipyridyl and the pharmaceutically acceptable salts thereof for preparing a medicament for treating rosacea. 6,6′-Dimethyl-2,2′-dipyridyl, 5,5′-dimethyl-2,2′-dipyridyl, 4,4′-dimethyl-2,2′-dipyridyl and the pharmaceutically acceptable salts thereof may be used for the treatment of rosacea.

The compound of formula (I), preferably chosen from 6,6′-dimethyl-2,2′-dipyridyl, 5,5′-dimethyl-2,2′-dipyridyl, 4,4′-dimethyl-2,2′-dipyridyl and the pharmaceutically acceptable salts thereof may thus be formulated in medicaments, referred to equally as “pharmaceutical compositions” subsequently, for human use. Said compositions comprise, in a pharmaceutically acceptable medium, at least one compound of formula (I).

The expression “pharmaceutically acceptable medium” means a medium that is compatible with the skin, the mucous membranes and the integuments.

The pharmaceutical composition that can be used according to the invention is administered topically.

Preferably, the compound of formula (I), preferably chosen from 6,6′-dimethyl-2,2′-dipyridyl, 5,5′-dimethyl-2,2′-dipyridyl, 4,4′-dimethyl-2,2′-dipyridyl and the pharmaceutically acceptable salts thereof, is present in a pharmaceutical composition for topical application.

The expression “topical application” means an application to the skin, the mucous membranes and/or the integuments.

The pharmaceutical composition according to the invention comprises from 0.001% to 10% by weight of compound(s) of formula (I) or salts thereof relative to the total weight of the composition. Preferably, the pharmaceutical composition according to the invention contains from 0.1% to 5% by weight of compound(s) of formula (I) or salts thereof relative to the total weight of the composition.

The topical pharmaceutical composition may be in liquid, pasty or solid form, and more particularly in the form of an ointment, a cream, a milk, an unguent, a powder, an impregnated pad, a syndet, a wipe, a solution, a gel, a spray, a foam, a suspension, a lotion, a stick, a shampoo or a cleansing base. It may also be in the form of a suspension of microspheres or nanospheres or lipid or polymer vesicles or a polymer patch and a hydrogel allowing controlled release. This pharmaceutical composition for topical application may be in anhydrous form, in aqueous form or in the form of an emulsion.

In one preferred variant of the invention, the pharmaceutical composition for topical application is in the form of a solution, a gel or an emulsion.

When the pharmaceutical composition according to the invention is in the form of an emulsion, it comprises at least one surfactant. An emulsion comprises a mixture of two immiscible liquids, one of which is dispersed in the other in the form of fine droplets (micelles); the dispersion is stabilized owing to the action of surfactants that modify the structure and the ratio of forces at the interface, and therefore increase the stability of the dispersion by decreasing the interfacial tension energy.

Surfactants are amphiphilic compounds which possess a hydrophobic portion that has an affinity for oil and a hydrophilic portion that has an affinity for water, thus creating a link between the two phases.

Surfactants thus stabilize oil/water emulsions by becoming adsorbed at the interface and by forming lamellar layers of liquid crystals. The surfactant may be ionic (anionic, cationic or amphoteric), or nonionic.

Among the surfactants that can be used according to the invention, examples that may be mentioned include: glyceryl/PEG100 stearate sold under the name Arlacel 165FL by the company Uniqema or under the name Simulsol 165 by the company SEPPIC, polyoxyethylenated fatty acid esters such as Arlatone 983 from the company Uniqema or the polyoxyethylenated stearyl alcohol (2) sold under the name Brij72 combined with the polyoxyethylenated stearyl alcohol (21) sold under the name Brij721 by the company Uniqema, sorbitan esters such as sorbitan oleate sold under the name Arlacel 80 by the company ICI or sold under the name Crill 4 by the company Croda, sorbitan sesquioleate sold under the name Arlacel 83 by the company ICI or sold under the name Montane 83 by the company SEPPIC, or else sorbitan isostearate; or else ethers of fatty alcohols.

The pharmaceutical composition according to the invention advantageously comprises up to 15% by weight, preferably from 2% to 12% by weight and more particularly from 2% to 6% by weight of suitable surfactant relative to the total weight of the composition.

Preferably, when the pharmaceutical composition is in the form of an emulsion, it is of water-in-oil or oil-in-water type. This type of emulsion comprises at least one lipophilic phase, one water-containing aqueous phase, and one surfactant.

The pharmaceutical composition in the form of an emulsion thus preferably comprises:

a) a lipophilic phase comprising fatty substances;
b) at least one surfactant;
c) at least one compound chosen from the compounds of formula (I) and salts thereof;
d) optionally one or more solvents and/or pro-penetrants of the compound of formula (I) or salts thereof;
e) and water.

The lipophilic phase of the pharmaceutical composition according to the invention may comprise, for example, plant, mineral, animal or synthetic oils, silicone oils, Guerbet alcohols, or other fatty substances and mixtures thereof.

As examples of mineral oils, mention may, for example, be made of liquid paraffins of various viscosities, such as Primol 352, Marcol 82 and Marcol 152 sold by the company Esso.

As plant oils, mention may be made of sweet almond oil, palm oil, soybean oil, sesame oil and sunflower oil.

As animal oils, mention may be made of lanolin, squalene, fish oil and mink oil.

As synthetic oils, mention may be made of esters such as cetearyl isononanoate sold in particular under the name Cetiol SN by the company Cognis France, diisopropyl adipate, such as the product sold under the name Ceraphyl 230 by the company ISF, isopropyl palmitate, such as the product sold under the name Crodamol IPP by the company Croda, and caprylic/capric triglyceride, such as Miglyol 812 sold by the company Huls/Lambert Rivière.

As silicone oils, mention may be made of a dimethicone such as the product sold under the name Dow Corning 200 fluid, a cyclomethicone such as the product sold under the name Dow Corning 244 fluid by the company Dow Corning or the product sold under the name Mirasil CM5 by the company SACI-CFPA.

As other fatty substances, mention may be made of fatty acids such as stearic acid, fatty alcohols such as stearyl alcohol, cetostearyl alcohol and cetyl alcohol or derivatives thereof, waxes such as beeswax, carnauba wax and candelilla wax and also gums, in particular silicone gums.

The ingredients of the lipophilic phase will be able to be chosen in a varied manner by a person skilled in the art so as to prepare a composition having the desired properties, for example in terms of consistency or texture.

The lipophilic phase of the emulsion according to the invention may be present in a content of between 3% and 50% by weight and preferably between 5% and 20% by weight relative to the total weight of the composition.

By way of example of a solvent and/or pro-penetrant of the compounds of formula (I) or salts thereof, mention will preferably be made of propylene glycol, alcohols of ethanol, isopropanol or butanol type, N-methyl-2-pyrrolidone or DMSO, polysorbate 80, phenoxyethanol and mixtures thereof.

The pharmaceutical composition in the form of an emulsion according to the invention preferably comprises from 0.1% to 20%, and preferably from 1% to 10% by weight relative to the total weight of the composition, of a solvent and/or pro-penetrant of the compounds of formula (I) or salts thereof.

The emulsion according to the invention also comprises an aqueous phase in an amount of between 30% and 95%, and preferably between 60% and 80% by weight relative to the total weight of the composition. This aqueous phase comprises water, preferably purified water.

The pharmaceutical composition according to the invention may also be in the form of a gel; it then comprises one or more gelling compounds, in an amount ranging from 0.01% to 5%, preferably from 0.1% to 3% by weight relative to the total weight of the composition.

Among the gelling agents that can be used in the pharmaceutical composition according to the invention, mention may be made of carboxyvinyl polymers (carbomers) and, as non-limiting examples of carbomers, Carbopol 981, Carbopol ETD 2020, Carbopol 980, Carbopol Ultrez 10 NF and Pemulen TR1 which are sold by the company Noveon. Mention may also be made of cellulose derivatives such as, for example, hydroxypropyl methyl cellulose, or hydroxyethyl cellulose; xanthan gums, aluminium/magnesium silicates such as Veegum K or Veegum Ultra sold by Vanderbilt, guar gums and the like, polyacrylamides such as the polyacrylamide/isoparaffin C13-14/laureth-7 mixture such as, for example, the one sold by the company SEPPIC under the name Sepigel 305, the acrylamide/AMPS copolymer as a 40% dispersion in isohexadecane sold under the name Simulgel 600PHA, or the family of modified starches such as Structure Solanace sold by National Starch, or mixtures thereof.

When the pharmaceutical composition is in the form of a solution, it comprises, besides the compounds of formula (I) or salts thereof, an aqueous phase or an oily phase, and optionally one or more solvents and/or pro-penetrants of the compounds of formula (I) as described above. The term “solution” is understood to mean a single-phase composition that is liquid at room temperature (25° C.), in which the compound of formula (I) or salts thereof is (are) dissolved.

The pharmaceutical composition according to the invention could also contain inert additives or combinations of these additives, such as:
preservatives;
stabilizers;
moisture regulators;
pH regulators;
osmotic pressure modifiers;
UV-A and UV-B screening agents;
and antioxidants.

Needless to say, a person skilled in the art will take care to select the optional compound(s) to be added to these pharmaceutical compositions such that the advantageous properties intrinsically associated with the present invention are not, or are not substantially, adversely affected by the envisaged addition.

These additives may be present in the pharmaceutical composition from 0.001% to 20% by weight relative to the total weight of the composition.

The results of the activity, on MMPs, of the compounds (I) and salts thereof will now be given by way of illustration and without being in any way limiting in nature.

The activity of MMPs of MMP-1, MMP-2, MMP-9, MMP-12, MMP-13 and TACE was measured using human recombinant proteins. The activity of MMP-8 was measured in human neutrophiles. After incubation with the corresponding substrate, the reaction product of each MMP was detected by fluorometry. The results are expressed as specific control activity percentages ((specific activity measured/specific control activity)×100) obtained in the presence of the compounds tested.

MMP activity (% of control values ± SD)

| | 4,4′-dimethyl- | 5,5′-dimethyl- | 6,6′-dimethyl- |
|---|---|---|---|
| | 2,2′-dipyridyl | 2,2′-dipyridyl | 2,2′-dipyridyl |
| | (10 μM) | (10 μM) | (10 μM) |
| MMP-1 | 94.3 ± 0.1 | 96.1 ± 0.3 | 98.8 ± 0.7 |
| MMP-2 | 100.8 ± 6.0 | 86.4 ± 2.2 | 95.9 ± 7.5 |
| MMP-8 | 86.5 ± 7.4 | 88.3 ± 6.7 | 95.0 ± 6.5 |
| MMP-9 | 88.5 ± 3.4 | 86.0 ± 3.4 | 101.0 ± 3.3 |
| MMP-12 | 96.3 ± 1.0 | 93.4 ± 0.4 | 96.3 ± 1.0 |
| MMP-13 | 90.0 ± 4.1 | 89.4 ± 1.2 | 96.2 ± 2.6 |
| MMP-14 | 90.8 ± 4.7 | 90.2 ± 4.1 | 84.3 ± 3.0 |
| TACE | 106.4 ± 1.2 | 100.8 ± 2.1 | 100.3 ± 0.2 |

The results of this study indicate that 4,4′-dimethyl-2,2′-dipyridyl, 5,5′-dimethyl-2,2′-dipyridyl and 6,6′-dimethyl-2,2′-dipyridyl were able to inhibit the activity of the MMPs. The compounds tested particularly inhibit MMP-8 and MMP-9, matrix metalloproteinases known for being upregulated in patients suffering from rosacea. These compounds were less active on certain other MMPs, such as MMP-1, MMP-2, MMP-12, MMP-14 and MMP-13. These MMPs, the inhibition of which by tetracycline derivatives, such as doxycycline, has been demonstrated, tetracyclines being known for being effective in the treatment of rosacea. Moreover, the compounds had no activity on TACE (TNF-alpha converting enzyme), the matrix metalloproteinase not being known for being involved in rosacea.

1. A method of treating rosacea, the method comprising administrating an effective amount of a compound of formula (I) below: 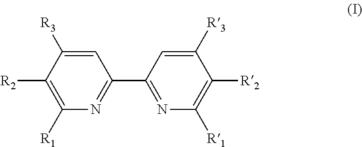
in which R1, R2, R3, R′1, R′2 and R′3 each independently represent a hydrogen atom or a linear or branched alkyl radical having from 1 to 6 carbon atoms, or a pharmaceutically acceptable salt thereof, to an individual subject in need thereof.

2. The method as defined by claim 1, wherein R1, R2, R3, R′1, R′2 and R′3 each independently represent a hydrogen atom or a methyl, ethyl, propyl, isopropyl, n-propyl, butyl, isobutyl or n-butyl radical.

3. The method as defined by claim 1, wherein at least one of the radicals R1, R2 and R3, and at least one of the radicals R′1, R′2 and R′3, each represent a methyl radical.

4. The method as defined by claim 1, wherein the compound is selected from the group consisting of 6,6′-dimethyl-2,2′-dipyridyl, 5,5′-dimethyl-2,2′-dipyridyl, 4,4′-dimethyl-2,2′-dipyridyl and pharmaceutically acceptable salts thereof.

5. The method as defined by claim 1, wherein the pharmaceutically acceptable salt of the compound is selected from the group consisting of a salt with hydrochloric acid, sulphuric acid, phosphoric acid, nitric acid, carbonic acid, boric acid, sulphamic acid, hydrobromic acid, acetic acid propionic acid, butyric acid, tartaric acid, maleic acid, hydroxymaleic acid, fumaric acid, citric acid, lactic acid, mucic acid, gluconic acid, benzoic acid, succinic acid, oxalic acid, phenylacetic, methanesulphonic, toluenesulphonic, benzenesulphonic, salicylic, sulphanilic, aspartic acid, glutamic acid, edetic acid, stearic acid, palmitic acid, oleic acid, lauric acid, panthotenic acid, tannic acid, ascorbic acid and valeric acid.

6. The method as defined by claim 1, wherein the compound is present in a pharmaceutical composition for topical application.

7. The method as defined by claim 6, wherein the pharmaceutical composition is in the form of a solution, gel or emulsion.

8. The method as defined by claim 6, wherein the compound is present in an amount from 0.001% to 10% by weight relative to the total weight of the composition.

9. The method as defined by claim 1 wherein the rosacea being treated is a subtype of rosacea selected from the group consisting of erythematotelangiectatic rosacea, papulopustular rosacea, phymatous rosacea and ocular rosacea.
